(19) United States
(12) Patent Application Publication
(10) Pub. No.: US 2012/0083597 A1
(43) Pub. Date: Apr. 5, 2012

(54) Enrichment of Low Molecular Weight DNA

(76) Inventors: Patricia Okamoto, Shrewsbury, MA (US); Jan Lawrence Godoski, JR., Seattle, WA (US); Thomas Scholl, Westborough, MA (US)

(21) Appl. No.: 13/245,265

(22) Filed: Sep. 26, 2011

Related U.S. Application Data
(60) Provisional application No. 61/389,042, filed on Oct. 1, 2010.

(51) Int. Cl. C07H 1/08

(52) U.S. Cl. 536/25.4

(57) ABSTRACT

The present invention provides, among other things, a simple, reproducible, and cost-effective method for enriching fetal or other low molecular weight nucleic acids in a biological sample. In certain embodiments, methods are provided for enriching fetal nucleic acids (e.g., fetal DNAs), typically comprising steps of adding a polymer such as PEG to a heterogeneous biological sample containing fetal DNA and high molecular weight non-fetal DNA such that the PEG precipitates substantially the high molecular weight non-fetal DNA, and purifying the fetal DNA from supernatant, thereby enriching the fetal DNA.

### PRIOR RELATED APPLICATIONS

This application claims priority to U.S. Provisional Application No. 61/389,042, filed Oct. 1, 2010, which is hereby incorporated by reference in its entirety.

### BACKGROUND

In a complex biological sample, nucleic acids of different cell or tissue origin may be characterized by differences in size or molecular weight. Accurate analysis of nucleic acids from particular cell types of interest has important clinical implications. For example, cell free fetal DNA in maternal circulation typically has lower molecular weight as compared to maternal DNA. Molecular analysis of cell free fetal DNA in a maternal sample has been shown to be a promising approach in non-invasive prenatal diagnosis of fetal aneuploidy, other fetal genetic abnormalities, and pregnancy complications. Some existing diagnostic methods and techniques typically perform well in clinical cases where the fraction of cell free fetal DNA in maternal plasma exceeds 25%. However, such levels of fetal DNA are rarely reached. If they are reached at all, they are typically reached only late in pregnancy when a therapeutic intervention is no longer an option. It has been observed that the fraction of cell free fetal DNA in maternal plasma varies between 0% and 5-10% in the first trimester of pregnancy between 4 and 13 weeks of gestation. To reach clinically useful accuracy in the first trimester of pregnancy, a significant enrichment of the fetal material is usually required for any of the currently developed assays. Therefore, significant efforts have been made to develop various methods for enriching fetal DNA. For example, methods based on size fractionation by electrophoresis have been developed. However, such methods are typically labor-intensive and have unpredictable yields. The average fetal DNA yield from some existing methods is as low as 1%. Therefore, what is needed is an improved method for enriching low molecular weight DNA, such as fetal DNA, in a complex sample.

### SUMMARY OF THE INVENTION

The present invention provides a simple, efficient, and cost-effective method for enriching low molecular weight DNA from complex biological samples. In particular, the present invention encompasses the recognition that selective size fractionation by a polymer such as polyethylene glycol (PEG) can be used to effectively precipitate high molecular weight DNA (e.g., maternal DNA) in a heterogeneous biological sample (e.g., maternal sample), leaving behind low molecular weight DNA (e.g., cell-free fetal DNA) in the supernatant. Low molecular weight DNA (e.g., fetal DNA) can then be purified or captured from the supernatant subsequently. Surprisingly, this simple method provides unexpectedly high yield of low molecular weight DNA (e.g., fetal DNA). As described in the Examples, the average yield of fetal DNA can be greater than 80%. The present invention may be used to enrich any type of small molecular weight nucleic acids and is particularly useful for enriching fetal DNA in a maternal sample. Thus, the present invention provides, among other things, a significant improvement in the non-invasive prenatal diagnostic field.

In one aspect, the present invention provides a method for enriching low molecular weight DNA comprising adding polyethylene glycol (PEG) to a heterogeneous biological sample containing low molecular weight DNA and high molecular weight DNA such that the PEG precipitates substantially the high molecular weight DNA; and purifying the low-molecular weight DNA from supernatant, thereby enriching the low molecular weight DNA. In some embodiments, the low molecular weight DNA has a size less than approximately 1 kb (e.g., less than approximately 750 bp, 500 bp, 450 bp, 400 bp, 350 bp, 300 bp, 250 bp, 200 by or 150 bp). In some embodiments, the high molecular weight DNA has a size greater than approximately 1 kb (e.g., greater than approximately 1.5 kb, 2.0 kb, 2.5 kb, 3 kb, 3.5 kb, 4.0 kb, 4.5 kb, or 5.0 kb). In some embodiments, the low molecular weight DNA is fetal DNA. In some embodiments, the high molecular weight DNA comprises maternal DNA. In certain embodiments, the present invention provides a method for enriching fetal DNA comprising adding polyethylene glycol (PEG) to a heterogeneous biological sample containing fetal DNA and high molecular weight non-fetal DNA wherein the PEG or PEG-like polymer precipitates substantially the high molecular weight non-fetal DNA; and purifying the fetal DNA from supernatant, thereby enriching the fetal DNA

In some embodiments, the purifying step comprises precipitating the low molecular weight DNA. In some embodiments, the purifying step comprises capturing the low molecular weight DNA with a solid support (e.g., magnetic beads). In some embodiments, a method according to the present invention further includes a step of collecting the supernatant by removing the precipitated high molecular weight DNA.

In some embodiments, the PEG is added such that the PEG is present in the heterogeneous biological sample at a concentration ranging from approximately 3-60% (e.g., from 5-20%, 5-12%, 5-10%). In some embodiments, the PEG is added such that the PEG is present in the heterogeneous biological sample at a concentration of approximately 8.3%. In some embodiments, the PEG is added such that the PEG is present in the heterogeneous biological sample at a concentration of approximately 10%. In some embodiments, the PEG suitable for the present invention has an average molecular weight ranging from approximately 1,500-8,000 daltons (e.g., approximately 3,000-8,000 daltons, approximately 3,000-6,000 daltons, approximately 1,500-6,000 daltons, or approximately 6,000-8,000 daltons). In some embodiments, the PEG suitable for the present invention has an average molecular weight of approximately 6,000 daltons. In some embodiments, the PEG suitable for the present invention has an average molecular weight of approximately 8,000 daltons. In some embodiments, a method according to the present invention further includes adding a salt together with the PEG to the heterogeneous biological sample. In certain embodiments, the salt comprises at least one of MgCl₂, MgSO₄, NaCl, ZnSO4, ZnCl₂, CaCl₂, or combinations thereof. In some embodiments, the salt used in a method of the invention comprises at least one of MgCl₂, MgSO₄, ZnSO₄, ZnCl₂, CaCl₂, or combinations thereof. In other embodiments, other salts may be used. In some embodiments, the salt is present at a concentration ranging from approximately 1.5-50 mM.

In some embodiments, the salt used in a method of the present invention is NaCl. In some such embodiments, the salt is present at a concentration ranging from about 0.2-3.0 M (e.g., about 0.25 M-2.0 M). In some embodiments, a method according to the present invention further includes a step of incubating the heterogeneous biological sample with added PEG at a temperature ranging from approximately 0-37° C. (e.g., about 0-25° C., about 19-25° C., or about 19-37° C.). In some embodiments, the heterogeneous biological sample is incubated for about 0-90 minutes (e.g., about 1-30 minutes, about 0.5-60 minutes, about 10-90 minutes, about 10-60 minutes, about 10-30 minutes, about 30-60 minutes, about 30-90 minutes). In some embodiments, the heterogeneous biological sample is selected from the group consisting of cells, tissue, whole blood, plasma, serum, urine, stool, saliva, cord blood, chorionic villus sample, chorionic villus sample culture, amniotic fluid, amniotic fluid culture, transcervical lavage fluid, and combinations thereof. In other embodiments, other types of biological samples may be used. In some embodiments, the heterogeneous biological sample is a maternal whole blood, plasma, serum, or other blood fraction sample. In certain embodiments, the heterogeneous biological sample is a maternal plasma or serum sample.

In some embodiments, the low molecular weight DNA represents less than about 10% (e.g., less than 5%, 4%, 3%, 2%, 1%, 0.1%) of the total nucleic acid in the heterogeneous biological sample. In some embodiments, the low molecular weight DNA is enriched by more than about 1.5-fold (e.g., more than 2-fold, 2.5-fold, 3-fold, 3.5-fold, 4-fold, 4.5-fold or 5-fold). In some embodiments, the yield of enriched low molecular weight DNA is greater than about 50% (e.g., greater than 55%, 60%, 65%, 70%, 75%, 80%, 85%, 90%, 95%, 96%, 97%, 98%, or 99%).

It is contemplated that inventive methods described herein may be used to enrich various small molecular weight DNA populations in complex biological samples. The present invention is however particularly useful for enriching fetal DNA in a maternal sample. In some embodiments, the present invention provides a method for enriching fetal DNA, comprising adding polyethylene glycol (PEG) to a heterogeneous biological sample containing fetal DNA and high molecular weight non-fetal DNA such that the PEG precipitates substantially the high molecular weight non-fetal DNA; and purifying the fetal DNA from supernatant, thereby enriching the fetal DNA.

In this application, the use of “or” means “and/or” unless stated otherwise. As used in this application, the term “comprise” and variations of the term, such as “comprising” and “comprises,” are not intended to exclude other additives, components, integers or steps.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIGS. 1A and 1B show exemplary results from a proof-of-principle experiment on mixtures of female genomic DNA spiked with a DNA ladder and a PCR-amplified fragment of the sex-determining region Y (SRY) gene (See Example 1). Low molecular mass DNA in the supernatant (FIG. 1A) and high molecular weight mass DNA in the pellet (FIG. 1B) can be seen in PEG-precipitated (8.3% PEG) DNA resolved on a 2% agarose gel.

FIG. 2 shows exemplary results from quantitative real-time PCR experiments for SRY DNA in supernatants after PEG precipitation of DNA mixtures spiked with SRY fragments (See Example 1). Error bars represent standard deviation in experiments run in triplicate.

FIG. 3 shows exemplary enrichment of the fetal fraction in maternal plasma samples by PEG precipitation methods of the present invention. The percent mean fetal fraction in the sample before PEG precipitation and in the supernatant after PEG precipitation were determined and plotted as shown. Error bars represent standard deviation from nine specimens.

### DEFINITIONS

In order for the present invention to be more readily understood, certain terms are first defined below. Additional definitions for the following terms and other terms are set forth throughout the specification.

As used herein, the term “allele” is used interchangeably with “allelic variant” and refers to a variant of a locus or gene. In some embodiments, different alleles or allelic variants are polymorphic.

As used herein, the term “amplification” refers to any methods known in the art for copying a target nucleic acid, thereby increasing the number of copies of a selected nucleic acid sequence. Amplification may be exponential or linear or both (i.e., have both a linear phase and an exponential phase). A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, and the like. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. Cell Probes 14:25-32 (2000)). This can be achieved using each pair of primers by reducing the concentration of one primer significantly relative to the other primer of the pair (e.g., 100 fold difference). Amplification by asymmetric PCR is generally linear. A skilled artisan will understand that different amplification methods may be used together.

As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

As used herein, the terms “approximately” and “about,” as applied to one or more values of interest, refers to a value that is similar to a stated reference value. In certain embodiments, the terms “approximately” or “about” are used interchangeably and refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “biological sample” encompasses any sample obtained from a biological source. A biological sample can, by way of non-limiting example, include blood (e.g., whole blood), serum, plasma, amniotic fluid, sera, urine, feces, epidermal sample, skin sample, cheek swab, sperm, amniotic fluid, cultured cells, bone marrow sample and/or chorionic villi. Convenient biological samples may be obtained, for example, by scraping cells from the surface of the buccal cavity. Cell cultures of any biological samples can also be used as biological samples, e.g., cultures of chorionic villus samples and/or amniotic fluid cultures such as amniocyte cultures. A biological sample can also be, e.g., a sample obtained from any organ or tissue (including a biopsy or autopsy specimen), can comprise cells (whether primary cells or cultured cells), medium conditioned by any cell, tissue, or organ, and tissue culture. In some embodiments, biological samples suitable for the invention are samples which have been processed to release or otherwise make available a nucleic acid for detection as described herein. Suitable biological samples may be obtained from a stage of life such as a fetus, young adult, adult (e.g., pregnant women), and the like. Fixed or frozen tissues also may be used. The terms “biological sample” and “biological specimen” are used interchangeably.

As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

As used herein, the term “fetal nucleic acid” refers to a nucleic acid whose origin is a fetal genome. In some embodiments, a fetal nucleic acid is present in a fetal cell. In some embodiments, a fetal nucleic acid is present in a cell-free fraction of a sample, e.g., a maternal sample. Such nucleic acid is also referred to as cell-free fetal nucleic acid. Typically, cell-free fetal nucleic acids are fragmented and have a lower average molecular mass than maternal nucleic acids.

As used herein, the term “gene” refers to a discrete nucleic acid sequence responsible for a discrete cellular (e.g., intracellular or extracellular) product and/or function. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. As used herein, the term “gene” can also include nucleic acids that do not encode proteins but rather provide templates for transcription of functional RNA molecules such as tRNAs, rRNAs, etc. Alternatively, a gene may define a genomic location for a particular event or function, such as a protein and/or nucleic acid binding site.

As used herein, the phrase “heterogeneous biological sample” refers to a biological sample that contains nucleic acids with different origins. For example, a heterogeneous biological sample may contain fetal nucleic acids and maternal nucleic acids.

As used herein, the term “high molecular weight DNA,” when used in connection with a maternal sample, generally refers to DNA that has a molecular weight greater than the average molecular weight of fetal DNA. In some embodiments, the term “high molecular weight DNA” refers to maternal DNA. In some embodiments, the term “high molecular weight DNA” refers to DNA having a size greater than approximately 1 kb. In some embodiments, the term “high molecular weight DNA” refers to DNA having a size greater than approximately 1.5 kb, 2.0 kb, 2.5 kb, 3 kb, 3.5 kb, 4.0 kb, 4.5 kb, or 5 kb.

As used herein, the term “isolated” refers to a substance or entity that has been (1) separated from at least some of the components with which it was associated when initially produced (whether in nature and/or in an experimental setting), and/or (2) produced, prepared, and/or manufactured by the hand of man. Isolated substances or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

The term “labeled” and the phrase “labeled with a detectable agent or moiety” are used herein interchangeably to specify that an entity (e.g., a nucleic acid probe, antibody, etc.) can be visualized, for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes, calorimetric labels, magnetic labels, haptens, molecular beacons, aptamer beacons, and the like.

As used herein, the phrase “low molecular weight DNA” is used interchangeably with “small molecular weight DNA” and generally refers to DNA that has a size less than about 1 kb. In some embodiments, the term “low molecular weight DNA” refers to DNA having a size less than about 750 bp, 500 bp, 450 bp, 400 bp, 350 bp, 300 bp, 250 bp, 200 bp, or 150 bp. In some embodiments, the term “low molecular weight DNA” refers to DNA that has a molecular weight less than the average molecular weight of maternal DNA. In some embodiments, the term “low molecular weight DNA” refers to cell-free fetal DNA. In some embodiments, the term “low molecular weight DNA” refers to viral genomic DNA.

As used herein, the term “maternal sample” refers to a biological sample obtained from a pregnant woman.

As used herein, the term “maternal nucleic acid” refers to a nucleic acid whose origin is a maternal genome.

As used herein, the term “polyethylene glycol” (abbreviated as “PEG”) refers to an oligomer or polymer of ethylene oxide. PEG typically has the following structure CAS number: 25322-68-3):

HO—(CH₂—CH₂—O)ₙ—H

wherein n is the average number of repeating oxyethylene units.

PEG compounds are often named by their average molecular weight, e.g., “PEG 400” would signify PEG having an average molecular weight of 400 daltons. PEG is also known as polyethylene oxide (PEO) or polyoxyethylene (POE) depending on its molecular weight. Polyethylene glycol may be known by its tradename CARBOWAX™.

As used herein, the term “subject” refers to a human or any non-human animal (e.g., mouse, rat, rabbit, dog, cat, cattle, swine, sheep, horse, or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “substantially” refers to the qualitative condition of exhibiting total or near total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “yield” refers to a ratio defined by the amount of low molecular weight DNA or fetal DNA recovered from a sample after performing a described enrichment method, as compared to the amount of molecular weight DNA or total fetal DNA present in the sample before performing such a method. In some embodiments, the method of interest is a method of enriching fetal DNA.

### DETAILED DESCRIPTION OF CERTAIN EMBODIMENTS

The present invention provides, among other things, a simple, reproducible, and cost-effective method for enriching fetal or other low molecular weight nucleic acids. In certain embodiments, provided are methods of enriching fetal nucleic acids (e.g., fetal DNAs), typically comprising steps of adding a polymer such as PEG to a heterogeneous biological sample containing fetal DNA and high molecular weight non-fetal DNA such that the PEG precipitates substantially the high molecular weight non-fetal DNA, and purifying the fetal DNA from supernatant, thereby enriching the fetal DNA.

As discussed in the Examples, when using methods of the invention, yield of fetal DNA is surprisingly high. Yield typically exceeds 50% (e.g., 60%, 70%, 80%, 90%, or more). Methods of the invention have resulted in enrichment of DNA by more than 1.5-fold, e.g., approximately 2-fold, 2.5-fold, 3-fold, 3.5-fold, 4-fold or more.

One embodiment of the invention is a method for enriching low molecular weight DNA comprising adding polyethylene glycol (PEG) or a PEG-like polymer to a heterogeneous biological sample containing low molecular weight DNA and high molecular weight DNA, wherein the low molecular weight DNA has a size less than about 500 base pairs (bp) and the high molecular weight DNA has a size greater than about 1 kb, and further wherein the PEG or PEG-like polymer precipitates substantially the high molecular weight DNA so that the low molecular weight DNA may be enriched by purifying it from the supernatant. In some embodiments, the low molecular weight DNA is fetal DNA. In some embodiments, the high molecular weight DNA is maternal DNA. In some embodiments, the low molecular weight DNA is precipitated from the supernatant. In other embodiments, the low molecular weight DNA is captured on a solid support. In certain embodiments, the solid support can be a magnetic bead. In other embodiments, the method further comprises a step of collecting the supernatant by removing the precipitated high molecular weight DNA.

In some embodiments, the PEG or PEG-like polymer is present in the heterogeneous biological sample at a concentration ranging from approximately 3-60%. In other embodiments, the PEG or PEG-like polymer is present in the heterogeneous biological sample at a concentration ranging from approximately 5-12% or approximately 5-10%. In other embodiments, the PEG or PEG-like polymer is present in the heterogeneous biological sample at a concentration of approximately 8.3% or 10%.

In some embodiments, the PEG or PEG-like polymer has an average molecular weight ranging from approximately 1,500-8,000 daltons. In one embodiment, the PEG or PEG-like polymer has an average molecular weight of approximately 6,000 daltons. In some embodiments, the PEG or PEG-like polymer has an average molecular weight of approximately 8,000 daltons.

In some embodiments, the method further comprises adding a salt together with the PEG or PEG-like polymer to the heterogeneous biological sample. In some embodiments, the salt may be MgCl₂, MgSO₄, NaCl, ZnSO₄, ZnCl₂, CaCl₂, or combinations thereof. In some embodiments, the salt is present at a concentration ranging from approximately 1.5-50 mM. In certain embodiments, the salt used in a method of the present invention is NaCl. In some such embodiments, the NaCl is present at a concentration ranging from about 0.2-3.0 M

In some embodiments, the method comprises incubating the heterogeneous biological sample with the PEG or PEG-like polymer at a temperature ranging from 0-37° C. In one embodiment, the heterogeneous biological sample is incubated with the PEG or PEG-like polymer at a temperature ranging from 19-25° C. In some embodiments, the heterogeneous biological sample is incubated for about 10-30 minutes.

### Samples and Preparation Thereof

Methods of the invention are typically performed on any samples including complex biological samples. As used herein, complex biological samples refer to heterogeneous biological samples containing nucleic acids (e.g., DNA) of different cell or tissue origin. In some embodiments, heterogeneous samples contain fetal nucleic acids and high molecular weight non-fetal nucleic acids (e.g., maternal DNA). In some embodiments, heterogeneous samples contain low molecular weight nucleic acids and high molecular weight nucleic acids. In some such embodiments, low molecular weight nucleic acids have a size of less than approximately 1 kb, (e.g., less than approximately 900 bp, 800 bp, 700 bp, 600 bp, 500 bp, 450 bp, 400 bp, 350 bp, 300 bp, 250 bp, 200 bp, 150 bp, 100 bp, or less) and high molecular weight nucleic acids have a size of more than about 1 kb (e.g., more than 1.5 kb, 2 kb, 2.5 kb, 3 kb, 3.5 kb, 4 kb, 4.5 kb, 5 kb, 6 kb, 7 kb, 8 kb, 9 kb, 10 kb, or more). In some embodiments, the low molecular weight nucleic acids has a size less than about 300 bp.

The present invention may be used to enrich low molecular weight DNA in a heterogeneous biological sample, in which the low molecular weight DNA to be enriched constitutes less than about 10% (e.g., less than 9%, 8%, 7%, 6%, 5%, 4%, 3%, 2%, 1%, 0.1%) of the total nucleic acids in the heterogeneous biological sample. In some embodiments, the low molecular weight nucleic constitutes less than 5% of the total nucleic acid in the heterogeneous biological sample. In certain embodiments, the low molecular weight nucleic constitutes less than 1% or less than 0.1% of the total nucleic acid in the heterogeneous biological sample.

In embodiments wherein heterogeneous samples contain fetal nucleic acids, at least some fetal nucleic acids may have a low molecular weight and corresponding small size, e.g., less than about 500 bp, 450 bp, 400 bp, 350 bp, or 300 by in size. In some embodiments, fetal nucleic acids represent less than about 10%, 5%, 1%, or 0.1%, of the total nucleic acids in the heterogeneous biological sample.

The present invention provides methods that in some embodiments, result in the low molecular weight nucleic acids being enriched by more than about 1.5-fold. In certain embodiments, the low molecular weight nucleic acids are enriched by approximately 2-fold. In some embodiments, the yield of the enriched low molecular weight nucleic acids is greater than about 50%. In certain embodiments, the yield is greater than about 80%.

Heterogeneous biological samples include, but are not limited to, cells, tissue, whole blood, plasma, serum, urine, stool, saliva, cord blood, chorionic villus samples, amniotic fluid, and transcervical lavage fluid. Cell cultures of any of the afore-mentioned heterogeneous biological samples also may be used in accordance with inventive methods, for example, chorionic villus cultures, amniotic fluid and/or amniocyte cultures, blood cell cultures (e.g., lymphocyte cultures), etc.

In certain embodiments, the heterogeneous biological sample is a maternal sample. Any of a variety of maternal samples may be suitable for use with methods disclosed herein. Generally, any maternal samples containing both fetal and maternal nucleic acids may be used. In some embodiments, a suitable maternal sample is obtained from a pregnant woman by a non-invasive method. For example, a suitable maternal sample can be a maternal blood, serum, or plasma sample obtained from a pregnant woman. In particular embodiments, a suitable maternal sample is maternal blood (e.g., peripheral venous blood).

Suitable maternal samples may be obtained from individuals at various stages of pregnancy (e.g., during first, second, or third trimester). In some embodiments, a suitable maternal sample is obtained during the first trimester, for example, between about 2-13 weeks (e.g., between about 6-13 weeks, between about 8-13 weeks, between about 9-13 weeks) of gestation. Typically, suitable maternal samples are obtained from individuals with a normal pregnancy. In some embodiments, a suitable maternal sample is obtained from one individual. In some embodiments, a suitable maternal sample is a pooled sample from multiple individuals.

In some embodiments, total DNA is prepared from a maternal sample. In some embodiments, cell-free DNA is prepared from a maternal sample. Various methods and kits for preparing total DNA or cell-free DNA are available in the art and can be used to practice the present invention. For example, nucleic acid can be extracted from a maternal sample by a variety of techniques such as those described by Maniatis et al., Molecular Cloning: A Laboratory Manual, Cold Spring Harbor, N.Y., pp. 280-281 (1982). Exemplary commercial kits that can be used to prepare cell-free DNA from maternal samples include, but are not limited to, QIAamp DNA Blood Midi Kit (Qiagen), High Pure PCR Template Preparation kit (Roche Diagnostics), and MagNA Pure LC (Roche Diagnostics).

Various amounts of maternal samples can be used. In some embodiments, a suitable maternal sample contains total or cell-free DNA with more than about 1 (e.g., more than 2, 5, 10, 15, 20, 25, 50, 100, 200, 500, 1,000, 5,000, or 10,000) genomic equivalents. It is contemplated that 10-20 ml of maternal blood contains about 10,000 genome equivalents of total DNA during first trimester. Thus, in some embodiments, a suitable maternal sample may contain about 20 ml, 15 ml, 10 ml, 5 ml, 4 ml, 3 ml, 2 ml, 1 ml, 0.5 ml, 0.1 ml, 0.01 ml, or 0.001 ml of maternal blood.

In some embodiments, maternal samples are treated in a manner to reduce background contamination of maternal DNA. For example, cellular apoptosis may contribute to maternal DNA contamination in the cell-free fraction. Reducing the contribution by apoptosis may be accomplished, for example, by gentler processing and handling as discussed below. In some embodiments, maternal samples are treated in a manner to facilitate keeping maternal DNA substantially intact. By “substantially intact,” it is meant that maternal DNA is substantially unfragmented and/or in fragments larger than approximately 1 kb (e.g., larger than 1.5 kb, 2 kb, 3 kb, 4 kb, 5 kb, or more) in size. For example, samples may be handled gently and/or quickly in order to avoid apoptosis or cell lysis and to prevent DNA shearing. In some embodiments, plasma and cellular components of blood sample are separated by gentle centrifugation. In some embodiments, fragments may be further treated such that the ends of the different fragments all contain the same DNA sequence. Fragments with universal ends can then be amplified in a single reaction with a single pair of amplification primers. Fragments with universal ends may also be captured onto a solid support by universal capturing probes. In some embodiments, to obtain unbiased quantification, no cloning or amplification is performed on nucleic acids in maternal samples before they are characterized by, e.g., sequencing, or hybridization.

It should be noted that, while the present description refers throughout to fetal DNA, fetal RNA found in maternal blood may be analyzed as well. As described in Ng et al., mRNA of placental origin is readily detectable in maternal plasma (Proc. Nat. Acad. Sci. 100(8): 4748-4753 (2003)). Both hPL, (human placental lactogen) and hCG (human chorionic gonadotropin) mRNA transcripts were detectable in maternal plasma. For example, mRNA encoding genes expressed in the placenta and present on the chromosome of interest can be used. In this case, RNase H minus (RNase H−) reverse transcriptases (RTs) can be used to prepare cDNA for detection.

### Polyethylene Glycol or PEG-Like Polymers

Polyethylene glycol (PEG) is a synthetic polymer of ethylene oxide, typically having the structure:

HO—(CH₂—CH₂—O)ₙ—H

wherein n is the average number of repeating oxyethylene units.

Both polydisperse (i.e., having a distribution of molecular weights) as well as monodisperse (i.e., having uniform molecular weight, and also known as “uniform” or “discrete”) PEGs are suitable for use in accordance with methods of the invention. PEGs are commercially available in a wide range of average molecular weights, typically with M_{w}<100,000 Da. Higher molecular weigh polymers are usually referred to as poly(ethylene oxide) (PEO) and may also be used in some embodiments of the invention. In some embodiments, a PEG having an average molecular weight (M_{w}) between about 1,500 and about 100,000 daltons (e.g., between about 1,500 and about 50,000 daltons, between about 1,500 and about 20,000 daltons, between about 3,000 to 8,000 daltons, etc.) is used. In some embodiments, a PEG having an average M_{w} of about 3,000 daltons or greater (e.g., PEG 4000 or higher, PEG 5000 or higher, PEG 6000 or higher, PEG 7000 or higher, PEG 8000 or higher, PEG 9000 or higher) is used. In some embodiments, a PEG having an average M_{w} of about 8,000 (PEG 8000) is used. In some embodiments, a mixture of PEGs of different molecular weights is used.

PEGs of various geometries may be used, including, but not limited to, branched PEGs, star PEGs, comb PEGs, and combinations thereof. Branched PEGs have PEG chains emanating from a central core group. In some embodiments, a branched PEG polymer has between 3 to 10 chains emanating from the core. Star PEGs typically have about 10-100 PEG chains emanating from a central core group. Comb PEGs typically have PEG chains grafted onto a polymer backbone.

PEGs can be synthesized using any of a variety initiators, including, but not limited to monofunctional (e.g., methyl ether), bifunctional, trifunctional, tetrafunctional, and other multifunctional initiators. PEGs are readily available commercially and may be used to practice the present invention. In some embodiments, PEGs are synthesized specifically for use with methods of the invention. Chain lengths, geometries, initiators, and other parameters can be chosen and/or controlled during synthesis as desired.

Derivatized PEGs may also be used in accordance with methods of the invention. For example, PEG derivatives such as PEG esters or PEG ethers can be used to covalently link DNA specific ligands that can then be used for DNA separation (See, e.g., Muller et al. (1981) “Polyethylene glycol derivatives of base and sequence specific DNA ligands: DNA interaction and application for base specific separation of DNA fragments by gel electrophoresis”, Nucleic Acids Research, 9(1) 95-119, the entire contents of which are incorporated by reference herein.) Any PEG derivative (e.g., chemically modified PEG) that can precipitate high molecular weight DNA can be used in accordance with methods of the invention.

### Other PEG-Like Polymers

Various other polymers also may be used to practice the present invention. Typically, polymers suitable for precipitation of high molecular weight DNA are neutral or cationic polymers. As used herein, such other polymers are referred to as PEG-like polymers. Non-limiting examples of suitable PEG-like polymers include polyamines (e.g., spermidine and spermine), polyaluminum chloride (PAC), dextrans (e.g., DEAE-dextran (diethyalaminoethyal-dextran, a cationic derivative of dextran)), polyacryl polymers, polyethyleneimine (PEI, polimin P), polyvinylamine (PVA), polyallylamine (PAA), polydimethylamino-ethylmethacrylate (PDMAEM), and poly-(N,N,N trimethylammonio)ethyl methacrylate chloride (PTMAEM), poly-l-lysine (PLL). (See, e.g., Raspaud et al. (1998) “Precipitation of DNA by Polyamines: A Polyelectrolyte Behavior,” Biophysical Journal, 74:381-393; Matsuzawa et al. (2003) “Study on DNA precipitation with a cationic polymer PAC (poly aluminum chloride,” Nucleic Acids Research Supplement, 3: 163-164; Maes et al. (1967) “Interaction between DEAE-dcxtran and nucleic acids,” Biochimica et Biophysica Acta (BBA)—Nucleic Acids and Protein Synthesis, 134(2):269-276; Kasyanenko et al. (2007) “DNA interaction with synthetic polymers in solution,” Structural Chemistry, 18(4):519-525; the entire contents of each of which are incorporated by reference herein.).

### Addition of PEG or PEG-Like Polymers to Biological Samples

In methods of the invention, PEGs or other PEG-like polymers are typically added to a biological sample to a final concentration of between about 3% and about 20% (e.g., about 3% to about 19%, about 4% to about 18%, about 5% to about 15%, about 5% to about 13%, about 5% to about 12%, or about 5% to about 10%). In some embodiments, PEGs are added to a final concentration of about 8.3% or about 10%.

In some embodiments, the resulting PEG- or PEG-like polymer-containing mixture is incubated at a particular temperature (e.g., between about 0° C. and about 37° C., between about 0° C. and about 25° C., between about 19° C. and about 25° C., between about 19° C. and about 37° C., or at room temperature) for a period of time (typically between about 5 minutes and overnight, e.g., from between approximately 5 minutes and 10 hours, 10 minutes and 16 hours, 10 minutes and 14 hours, between 10 minutes and 12 hours, 10 minutes and 10 hours, 10 minutes and 8 hours, 10 minutes and 6 hours, 10 minutes and 5 hours, 10 minutes and 4 hours, 10 minutes and 3 hours, 10 minutes and 2 hours, 10 minutes and 1 hour, 10-90 minutes, 10-60 minutes, or 10-30 minutes) to facilitate precipitation of higher molecular weight molecules (e.g., nucleic acids) in the biological sample. For example, incubation times may be about 5 minutes, 10 minutes, 15 minutes, 20 minutes, 25 minutes, 30 minutes, 45 minutes, 60 minutes, 1.5 hours, 2 hours, or longer. In some embodiments, a PEG- or PEG-like polymer-containing mixture is incubated for about 10 minutes at about 22-23° C. In some embodiments, a PEG- or PEG-like polymer-containing mixture is incubated for about 10-30 minutes at room temperature.

In some embodiments, PEG- or PEG-like polymer-mediated precipitation of higher molecular weight molecules is performed in the presence of one or more salts, which may be already present in the biological sample, introduced with the PEG or PEG-like polymer, and/or introduced after adding the PEG or PEG-like polymer. Non-limiting examples of suitable salts include MgCl₂, MgSO₄, NaCl, ZnSO₄, ZnCl₂, and CaCl₂. Any combination of such salts also may be used. Typically, magnesium, zinc, and calcium based salts are used at a concentration of between about 1.5 and about 50 mM (e.g., about 1.5 mM, 2, mM, 2.5 mM, 3 mM, 3.5 mM, 4 mM, 4.5 mM, 5 mM, 10 mM, 15 mM, 20 mM, 25 mM, 30 mM, 35 mM, 40 mM, 45 mM, or 50 mM), and NaCl is typically used at a concentration of between 0.2 and 3.0 M (e.g., about 0.2 M, 0.25 M, 0.3 M, 0.4 M, 0.5 M, 0.6 M, 0.7 M, 0.8 M, 0.9 M, 1.0 M, 1.1 M, 1.2 M, 1.3 M, 1.4 M, 1.5 M, 1.6 M, 1.7 M, 1.8 M, 1.9 M, 2 M, 2.1 M, 2.2 M, 2.3 M, 2.4 M, 2.5 M, 2.6 M, 2.7 M, 2.8 M, 2.9M, or 3.0 M).

In some embodiments, the PEG- or PEG-like polymer-containing mixture contains buffer components that are typically used in DNA solutions, such as Tris and/or EDTA (ethylenediaminetetraacetic acid). For example, in some embodiments, the final buffer of a PEG-containing mixture may include about 10-50 mM MgCl₂, about 5-10% PEG 8000, about 1-10 mM Tris, about pH 7.5-8.0, and about 0.1-1.0 mM EDTA.

### Purification of Low Molecular Weight Nucleic Acids

Low molecular weight nucleic acids (e.g., fetal nucleic acids such as fetal DNAs) are typically purified from the supernatants obtained after addition of the PEG or PEG-like polymer and precipitation. A variety of purification methods are known in the art and are suitable for use in accordance with methods of the invention.

In some embodiments, low molecular weight nucleic acids are purified (e.g., extracted) by precipitation from supernatants (e.g., after removing the precipitated high molecular weight nucleic acids) using standard molecular biology techniques for DNA extraction or precipitation. Typically, this precipitation step does not involve the use of PEG, or uses PEG in smaller amounts and/or uses PEGs of smaller average molecular weights as compared to PEGs used in the previous step. For example, low molecular weight nucleic acids can be precipitated from supernatants by adding about two volumes or more (e.g., about 2 or about 2.5) of an alcohol such as ethanol. Absolute (100%) ethanol is typically employed in this manner, although high percentage (e.g., at least 95%) ethanols may also be used. In some embodiments, the salt concentrations of supernatants are adjusted to facilitate precipitation. Salts such as sodium acetate (typically at a final concentration of about 0.3 M-0.4 M) or ammonium acetate (typically at a final concentration of about 2.0-2.5 M) are employed in this manner. After addition of alcohols and/or adjustment of salt concentrations, solutions are optionally incubated at a cold temperature (e.g., placed on ice and/or in a freezer at −10° C., −20° C., −70° C., −80° C., or lower). A DNA precipitate containing fetal nucleic acids can be isolated from the supernatant/alcohol solution, for example, by centrifugation.

In some embodiments, the step of purifying low molecular weight nucleic acids comprises capturing low molecular weight nucleic acids (e.g., DNA) in the supernatants using a solid support. In some embodiments, the low molecular weight nucleic acids may be purified directly from the supernatant without first removing the precipitated high molecular weight DNA. In some embodiments, the solid support comprises a bead (e.g., magnetic beads and/or paramagnetic beads) and bead-based separation methods are employed. Bead-based separation may be silica-based and/or charge-based. For example, DNA selectively binds to silica (e.g., coated on the surface of a magnetic bead) in the presence of chaotropic salts (e.g., guanidium HCl) and can be released by altering the salt concentration. Purification methods that do not rely on use of chaotropic salts are also suitable for use in methods of the present invention. Alternatively or additionally, positively charged beads attract negatively charged DNA. In some such charge-based methods, DNA is released from beads by altering the pH of the solution.

In some embodiments, the step of purifying comprises using solid phase reversible immobilization (SPRI). (See, e.g., Hawkins et al. (1995) Nucleic Acids Res. (23): 4742-4743, the entire contents of which are incorporated by reference herein.) SPRI purification methods typically employ paramagnetic beads coated with a carboxylate-modified polymer and allow elution without the use of chaotropic salts.

In some embodiments, the step of purifying comprises using a column, e.g., a filtration column or chromatography column. For example, hydroxyapatite (a form of calcium phosphate; also known as hydroxylapatite) columns may be employed.

### Additional Steps

In some embodiments, additional steps may be performed. Such steps may be performed at any time relative to the other steps, e.g., during, before, or after the step of adding PEG and/or during, before, or after the step of purifying low molecular weight DNA. Such steps may be performed, for example, to enhance the purity and/or increase the yield of the final enriched low molecular weight DNA.

In some embodiments, agents that denature proteins and/or disrupt nucleoprotein complexes are added to samples or mixtures. For example, phenol/chloroform mixtures (optionally including one or more stabilizing agents such as isoamyl alcohol) may be used. Additionally or alternatively, agents may be added to inactivate endogenous nucleases and therefore reduce the extent of degradation of nucleic acids in the sample. Non-limiting examples of agents employable in this manner include nuclease inhibitors (e.g., DNAse inhibitors) and chelating agents (e.g., EDTA and EGTA (ethylene glycol tetraacetic acid)). Such steps may be particularly desirable to keep high molecular weight DNAs such as maternal DNA large or intact (e.g., >1000 bp) to facilitate selective fractionation by PEG precipitation.

In some embodiments, nucleic acids are detectably labeled, e.g., to facilitate their purification and/or characterization in the subsequent diagnostic analysis.

### Applications

Methods of the invention may find use in diagnostic applications based on low molecular weight nucleic acids. For example, methods of the invention may be useful in diagnosing fetal conditions based on enriched fetal DNA from prenatal samples. In some embodiments, methods of the invention may be used to enrich nucleic acids from pathogens (e.g., virus, bacteria, fungi, parasites, among others) and cells associated with certain diseases, disorders and conditions (e.g., cancer, autoimmune diseases, infectious diseases, tissue or organ transplant, among others).

The following discussion provides non-limiting examples of diseases, disorders, or conditions whose diagnosis may be facilitated by methods of the invention. For example, fetal or other nucleic acids enriched by methods of the invention may be evaluated for the presence of mutations such as nucleic acid base substitutions, duplications, insertions, deletions and/or translocations. In some embodiments, fetal or other nucleic acids obtained by methods of the present invention are used in diagnostic methods that detect mutations associated with rare events in a biological sample. For example, enriched nucleic acids may be evaluated by methods that detect mutations in rare cells present in a biological sample. In some embodiments, such rare cells are cancer cells present in a biological sample (e.g., whole blood) from a patient. In some embodiments, such rare cells are fetal cells present in maternal blood. In some embodiments, such rare cells are pathogens associated with infectious diseases. In some embodiments, such rare cells are immune cells associated with autoimmune diseases or immunological conditions associated with transplant, and the like. Thus, the present invention can be used to enrich fetal or other nucleic acids for pre-natal diagnosis of fetal abnormalities and early diagnosis of cancer and other pathological conditions.

In some embodiments, enriched nucleic acid fractions are used in methods to determine relative amount of a target nucleic acid in comparison to a reference nucleic acid (e.g., to determine a ratio). For example, enriched nucleic acids may be used in methods that detect imbalance(s) of any chromosomes or a number of genetic loci implicated in genetic diseases. Thus, methods disclosed herein can facilitate detection of carriers, diagnosis of patients, prenatal diagnosis, and/or genotyping of embryos for implantation, etc. As appreciated by those of ordinary skill in the art, genetic diseases can follow any of a number of inheritance patterns, including, for example, autosomal recessive, autosomal dominant, sex-linked dominant, and sex-linked recessive.

In some embodiments, enriched fetal nucleic acids are used in diagnostic methods that detect genetic abnormalities that involve quantitative differences between maternal and fetal genetic nucleic acids. These genetic abnormalities include mutations that may be heterozygous and homozygous between maternal and fetal DNA, and aneuploidies. For example, a missing copy of chromosome X (monosomy X) results in Turner's Syndrome, while an additional copy of chromosome 21 results in Down Syndrome.

Other diseases such as Edward's Syndrome and Patau Syndrome are caused by an additional copy of chromosome 18, and chromosome 13, respectively. Diagnostic methods may detect a deletion, translocation, addition, amplification, transversion, inversion, aneuploidy, polyploidy, monosomy, trisomy including but not limited to trisomy 21, trisomy 13, trisomy 14, trisomy 15, trisomy 16, trisomy 18, trisomy 22, triploidy, tetraploidy, and sex chromosome abnormalities including but not limited to X0, XXY, XYY, and XXX.

Alternatively or additionally, specific genetic loci such as genes or portions thereof (e.g., exons, introns, promoters, or other regulatory regions) may be analyzed in enriched fetal nucleic acids obtained by methods of the present invention. Table 1 lists non-limiting examples of such genes and associated genetic diseases, disorders, or conditions. As understood by one of ordinary skill in the art, a gene may be known by more than one name. The listing in Table 1 does not exclude the existence of additional genes that may be associated with a particular disease. Applications of the present invention encompass diagnostic methods that examine those additional genes (including those that will be discovered in the future) associated with each particular diseases.

TABLE 1
Exemplary genes associated with genetic diseases, disorders or conditions

| Disease, Disorder or condition | Gene | Protein Product |
|---|---|---|
| Achondroplasia | FGFR3 | fibroblast growth factor receptor 3 |
| Adrenolcukodystrophy | ABCD 1 | ATP-binding cassette (ABC) |
| | | transporters |
| Alpha-1-antitrypsin deficiency | SERPINA1 | serine protease inhibitor |
| Alpha-thalassemia | HBA 1&.2 | hemoglobin alpha 1 &2 |
| Alport syndrome | COL4A5 | collagen, type IV, alpha 5 |
| Amyotrophic lateral sclerosis | SOD I | superoxide dismutasc 1 |
| Angelman syndrome | UBE3A | ubiquitin protein ligase E3A |
| Ataxia telengiectasia | ATM | ataxia telangiectasia mutated |
| Autoimmune polyglandular | AIRE | autoimmune regulator |
| syndrome | | |
| Bloom syndrome | BLM, RECQL3 | recQ3 helicase-like |
| Burkitt lymphoma | MYC | v-myc myelocytomatosis viral |
| | | oncogene homolog |
| Canavan disease | ASPA | aspartoacylase |
| Congenital adrenal hyperplasia | CYP21 | cytochrome P450, family 21 |
| Cystic fibrosis | CFTR | cystic fibrosis transmembrane |
| | | conductance regulator |
| Diastrophic dysplasia | SLC26A2 | sulfate transporter |
| Duchenne muscular dystrophy | DMD | Dystrophin |
| Familial dysautonomia | 1KBKAP | IKK complex-associated protein |
| | | (1KAP) |
| Familial Mediterranean fever | MEFV | Mediterranean fever protein |
| Fanconi anemia | FANCA, FANCB | (proteins involved in DNA repair) |
| | (FAAP95), FANCC, | |
| | FANCD1 (BRCA2), | |
| | FANCD2, FANCE, | |
| | FANCF, FANCG, | |
| | FANCI, FANCJ | |
| | (BRIP1), FANCL | |
| | (PHF9 and POG), | |
| | FANCM (FAAP250) | |
| Fragile X syndrome | FMR I | fragile X mental retardation 1 |
| Friedrich's ataxia | FRDA | Frataxin |
| Gaucher disease | GBA | glucosidase |
| Glucose galactose malabsorption | SGLT 1 | sodium-dependent glucose |
| | | cotransporter |
| Glycogen disease type I (GSD1) | G6PC (GSDIa) | glucose-6-phosphatase |
| | SLC37A4 | glucose-5-phosphate transporter 3, |
| | (GSDIb) | solute carrier family 37 member 4 |
| Gyrate atrophy | OAT | crnithine aminotransferase |
| Hemophilia A | F8 | coagulation factor VIII |
| Hereditary hemocrhomatosis | HFE | hemochromatosis protein |
| Huntington disease | HD | Tuntingtin |
| Immunodeficiency with hyper-IgM | TNFSF5 | humor necrosis factor member 5 |
| Lesch-Nyhan syndrome | HPRT 1 | hypoxanthine phosphoribotransferase |
| Maple syrup urine disease | BCKDHA | branched chain keto acid |
| (MSUD) | | dehydrogenase |
| Marfan syndrome | FBN1 | Fibrillin |
| Megalencephalic | MLC1 | (putative transmembrane protein) |
| leukoencephalopathy | | |
| Menkes syndrome | ATP7A | ATPase Cu++ transporting |
| Metachromatic leukodystrophy | ARSA | arylsulfatase A |
| (MLD) | | |
| Mucolipidosis IV (ML IV) | MCOLN 1 | Mucolipin-1 |
| Myotonic dystrophy | DMPK | myotonic dystrophy protein kinase |
| Nemaline myopathy | | |
| Neurofibromatosis | NF1, NF2 | neurofibromin |
| Niemann Pick disease (types A | SMPD1 | sphingomyelin phosphodiesterase 1, |
| and B type) | | acid lysosomal (acid |
| | | sphingomyelinase) |
| Niemann Pick disease (type C) | NPC1, NPC2 | Niemann-Pick disease, type Cl (an |
| | | integral membrane protein) and |
| | | Niemann-Pick disease, type C2 |
| Paroxysmal nocturnal | PIGA | phosphatidylinositol glycan |
| hemoglobinuria | | |
| Pendred syndrome | PDS | Pendrin |
| Phenylketonuria | PAH | phenylalanine hydroxylase |
| Refsum disease | PHYH | Phytanoyl-CoA hydroxylase |
| Retinoblastoma | RB | retinoblastoma I |
| Rett syndrome | MECP2 | methyl CpG binding protein |
| SCID-ADA | ADA | adenosine deaminase |
| (Severe combined | | |
| immunodeficiency-ADA) | | |
| SCID-X-linked | IL2RG | Interleukin-2-receptor, gamma |
| (Sever combined | | |
| immunodeficiency-X-linked) | | |
| Sickle cell anemia (also known as | HBB | hemoglobin, beta |
| beta-thalassemia) | | |
| Spinal muscular atrophy (SMA) | SMN1, | survival of motor neuron 1, |
| | SMN2 | Survival of motor neuron 2 |
| Tangier disease | ABCA1 | ATP-binding cassette A1 |
| Tay-Sachs disease | HEXA | hexosaminidase |
| Usher syndrome | MYO7A | myosin VIIA |
| (Also known as Hallgren | USH1C | Harmonin |
| syndrome, Usher-Hallgren | CDH23 | cadherin 23 |
| syndrome, rp-dysacusis syndrome | PCDH15 | protocadherin 15 |
| and dystrophia retinae dysacusis | USH1G | SANS |
| syndrome.) | USH2A | Usherin |
| | GPR98 | VLGRIb |
| | DFNB31 | Whirlin |
| | CLRN1 | clarin-1 |
| Von Hippcl-Lindau syndrome | VHL | elongin binding protein |
| Werner syndrome | WRN | Werner syndrome protein |
| Wilson's disease | ATP7B | ATPase, Cu++ transporting |
| Zellweger syndrome | PXR 1 | peroxisome receptor 1 |

Thus, methods of the invention can be applied in diagnostic methods that analyze one or more genes, including, but not limited to, genes identified in Table 1, or a portion thereof (e.g., coding (e.g., exon) or non-coding (e.g., intron, or regulatory) region). The sequences of the genes identified in Table 1 are known in the art and are readily accessible by searching in public databases such as GenBank using gene names and such sequences are incorporated herein by reference.

Although most genes are normally present in two copies per genome equivalent, a large number of genes have been found for which copy number variations exist between individuals. Copy number differences can arise from a number of mechanisms, including, but not limited to, gene duplication events, gene deletion events, gene conversion events, gene rearrangements, chromosome transpositions, etc. Differences in copy numbers of certain genes may have implications including, but not limited to, risk of developing a disease or condition, likelihood of progressing to a particular disease or condition stage, amenability to particular therapeutics, susceptibility to infection, immune function, etc. In addition to the genes listed in Table 1, nucleic acids obtained by methods of the invention may be used in diagnostic methods that are suitable for analyzing copy numbers at loci with such copy number variants. The Database of Genomic Variants, which is maintained at the website whose address is “http://” followed immediately by “projects.tcag.ca/variation” (the entire contents of which are herein incorporated by reference in their entirety), lists more than at least 38,406 copy number variants (as of Mar. 11, 2009). (See, e.g., Iafrate et al. (2004) “Detection of large-scale variation in the human genome” Nature Genetics. 36(9):949-51; Zhang et al. (2006) “Development of bioinformatics resources for display and analysis of copy number and other structural variants in the human genome.” 115(3-4):205-14; Zhang et al. (2009) “Copy Number Variation in Human Health, Disease and Evolution,” Annual Review of Genomics and Human Genetics. 10:451-481; and Wain et al. (2009) “Genomic copy number variation, human health, and disease.” Lancet. 374:340-350, the entire contents of each which are herein incorporated by reference).

Examples of diseases where the target sequence may exist in one copy in the maternal DNA (heterozygous) but if inherited from both parents cause disease in a fetus (homozygous), include sickle cell anemia, cystic fibrosis, hemophilia, and Tay Sachs disease. Enriched nucleic acids obtained by methods of the invention may be used in methods that distinguish genomes with one mutation from genomes with two mutations. Sickle-cell anemia is an autosomal recessive disease. Nine-percent of African-Americans are heterozygous, while 0.2% are homozygous recessive. The recessive allele causes a single amino acid substitution in the beta chain of hemoglobin.

Tay-Sachs Disease is an autosomal recessive resulting in degeneration of the nervous system. Symptoms manifest after birth. Children homozygous recessive for this allele rarely survive past five years of age. Sufferers lack the ability to make the enzyme N-acetyl-hexosaminidase, which breaks down the GM2 ganglioside lipid.

Another example is phenylketonuria (PKU), a recessively inherited disorder whose sufferers lack the ability to synthesize an enzyme to convert the amino acid phenylalanine into tyrosine. Individuals homozygous recessive for this allele have a buildup of phenylalanine and abnormal breakdown products in the urine and blood.

Hemophilia is a group of diseases in which blood does not clot normally. Factors in blood are involved in clotting. Hemophiliacs lacking the normal Factor VIII are said to have Hemophilia A, and those who lack Factor IX have Hemophilia B. These genes are carried on the X chromosome, so primers and probes may be used in the present method to detect whether or not a fetus inherited the mother's defective X chromosome, or the father's normal allele.

A listing of gene mutations for which the present method may be adapted is found at The GDB Human Genome Database, The Official World-Wide Database for the Annotation of the Human Genome Hosted by RTI International, North Carolina USA (www.gdb.org/gdb).

As mentioned above, the presently disclosed methods also may be used to enrich other (e.g., non-fetal) low molecular weight nucleic acids for other diagnostic applications. Non-limiting examples of such applications include diagnosis and/or detection of any conditions involving cellular apoptosis, such as early cancer detection, viral or bacterial infection, and autoimmune disease.

### EXAMPLES

The present invention may be better understood by reference to the following non-limiting examples.

### Example 1

### Enrichment of Low Molecular Weight DNA in Prepared Mixtures Using Peg Precipitation

The present Example demonstrates a proof-of-principle of inventive methods on prepared mixtures of DNA.

Female genomic DNA was spiked with increasing amounts of a 50 bp ladder (Invitrogen) and a 64 by PCR-amplified sex-determining region Y (SRY) fragment from the Y chromosome. The ladder was included to facilitate determining a limit, if any, of size fractionation after PEG precipitation.

PEG (MW 8000) was mixed to a final concentration of 8.3% or 10% with DNA mixtures in the presence of 10 mM MgCl₂. The mixture was incubated at 22-23° C. to selectively precipitate higher molecular weight DNA. High molecular weight DNA was pelleted by centrifugation at 16,000×g for 30 minutes. Supernatants were collected for further manipulations as described below, and the pellet containing high molecular weight DNA was resuspended in Tris-EDTA buffer, pH 8.

DNA was precipitated from supernatants according to standard molecular biology techniques. DNA precipitated from this second precipitation step (performed without PEG) was also resuspended in Tris-EDTA buffer, pH 8. DNA samples were resolved by electrophoresis through a 2% agarose gel and visualized by staining with ethidium bromide.

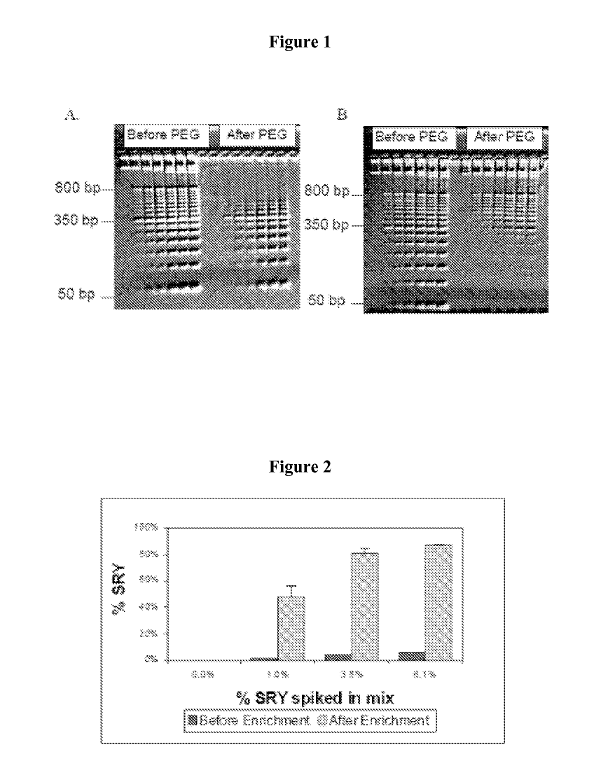

As shown in FIG. 1A, low molecular weight DNA is enriched in the supernatant after precipitation with 8.3% PEG. Size fractionation was achieved at approximately 300 bp to approximately 500 bp. Similar results were also observed with 10% PEG.

Enrichment of the spiked SRY sequence was also measured by quantitative real-time PCR. As shown in FIG. 2, quantitative real-time PCR results confirmed that SRY DNA was enriched in the supernatant after precipitation with 8.3% PEG.

### Example 2

### Enrichment of Fetal DNA in Maternal Plasma Using PEG Precipitation

The present Example demonstrated that PEG precipitation methods of the invention can be used to selectively fractionate and enrich male fetal DNA in maternal plasma. Maternal plasma was collected from pregnant subjects. DNA was extracted and precipitated from maternal plasma as described in Example 1, i.e., using a PEG-precipitation step to selectively precipitate high molecular weight DNA, followed by precipitation (without PEG) of the supernatant to obtain an enriched low molecular weight fraction.

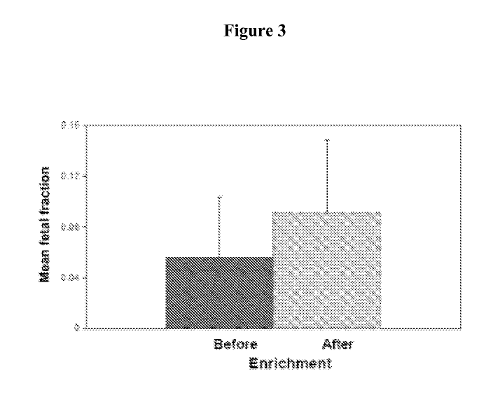

Fetal DNA yield from supernatants after PEG precipitation samples was determined by quantitative real-time PCR to detect the SRY gene (as performed in Example 1). The average fetal DNA yield from nine specimens was >80%, demonstrating that most of the fetal DNA can be recovered in the supernatant after PEG precipitation. The mean (average of 9 specimens) fetal fraction in the sample before PEG precipitation and in the supernatant after PEG precipitation as determined by quantitative real-time PCR were calculated (FIG. 3). (“Fetal fraction” as used herein refers to the amount of fetal DNA over the total amount of all DNAs (e.g., fetal and maternal) in a sample.) About half of the maternal fraction was removed by PEG precipitation, resulting in a mean enrichment of approximately 1.8-fold.

### INCORPORATION OF REFERENCES

All publications and patent documents cited in this application are incorporated by reference in their entirety to the same extent as if the contents of each individual publication or patent document were incorporated herein.

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method for enriching low molecular weight DNA comprising
(a) adding polyethylene glycol (PEG) or a PEG-like polymer to a heterogeneous biological sample containing low molecular weight DNA and high molecular weight DNA, wherein the low molecular weight DNA has a size less than about 500 bp and the high molecular weight DNA has a size greater than about 1 kb, and further wherein the PEG or PEG-like polymer substantially precipitates the high molecular weight DNA; and
(b) purifying the low molecular weight DNA from supernatant, thereby enriching the low molecular weight DNA.

2. The method of claim 1, wherein the low molecular weight DNA comprises fetal DNA.

3. The method of claim 2, wherein the high molecular weight DNA comprises maternal DNA.

4. The method of claim 1, wherein the purifying step comprises precipitating the low molecular weight DNA.

5. The method of claim 1, wherein the purifying step comprises capturing the low molecular weight DNA with a solid support.

6. The method of claim 5, wherein the solid support comprise a magnetic bead.

7. The method of claim 1, wherein the method further comprises a step of collecting the supernatant by removing the precipitated high molecular weight DNA.

8. The method of claim 1, wherein the PEG or PEG-like polymer is present in the heterogeneous biological sample at a concentration ranging from approximately 3-60%.

9. The method of claim 1, wherein the PEG or PEG-like polymer is present in the heterogeneous biological sample at a concentration ranging from approximately 5-12%.

10. The method of claim 1, wherein the PEG or PEG-like polymer is present in the heterogeneous biological sample at a concentration ranging from approximately 5-10%.

11. The method of claim 10, wherein the PEG or PEG-like polymer is present in the heterogeneous biological sample at a concentration of approximately 8.3%.

12. The method of claim 10, wherein the PEG or PEG-like polymer is present in the heterogeneous biological sample at a concentration of approximately 10%.

13. The method of claim 1, wherein the PEG or PEG-like polymer has a molecular weight ranging from approximately 1,500-8000 daltons.

14. The method of claim 1, wherein the PEG or PEG-like polymer has a molecular weight of approximately 6,000 daltons.

15. The method of claim 1, wherein the PEG or PEG-like polymer has a molecular weight of approximately 8,000 daltons.

16. The method of claim 1, wherein the method further comprises adding a salt together with the PEG or PEG-like polymer to the heterogeneous biological sample.

17. The method of claim 16, wherein the salt comprises at least one of MgCl₂, MgSO₄, NaCl, ZnSO₄, ZnCl₂, or CaCl₂.

18. The method of claim 17, wherein the salt is present at a concentration ranging from 1.5-50 mM.

19. The method of claim 16, wherein the salt is NaCl.

20. The method of claim 19, wherein the NaCl is present at a concentration ranging from 0.2-3.0 M.

21. The method of claim 1, wherein the method further comprises incubating the heterogeneous biological sample with the PEG or PEG-like polymer at a temperature ranging from 0-37° C.

22. The method of claim 21, wherein the temperature ranges from 19-25° C.

23. The method of claim 21, wherein the heterogeneous biological sample is incubated for about 10-30 minutes.

24. The method of claim 1, wherein the heterogeneous biological sample is selected from the group consisting of cells, tissue, whole blood, plasma, serum, urine, stool, saliva, cord blood, chorionic villus sample, chorionic villus sample culture, amniotic fluid, amniotic fluid culture, transcervical lavage fluid, and combinations thereof.

25. The method of claim 1, wherein the heterogeneous biological sample is a maternal blood, plasma, or serum sample.

26. The method of claim 1, wherein the low molecular weight DNA has a size less than about 300 bp.

27. The method of claim 1, wherein the low molecular weight represents less than about 5% of the total nucleic acid in the heterogeneous biological sample.

28. The method of claim 27, wherein the low molecular weight DNA represents less than about 1% of the total nucleic acid in the heterogeneous biological sample.

29. The method of claim 28, wherein the low molecular weight DNA represents less than about 0.1% of the total nucleic acid in the heterogeneous biological sample.

30. The method of claim 1, wherein the low molecular weight DNA is enriched by more than about 1.5-fold.

31. The method of claim 1, wherein the low molecular weight DNA is enriched by more than about 2-fold.

32. The method of claim 1, wherein the yield of enriched low molecular weight DNA is greater than 50%.

33. The method of claim 32, wherein the yield of enriched low molecular weight DNA is greater than 80%.

34. A method for enriching fetal DNA, comprising
adding polyethylene glycol (PEG) or a PEG-like polymer to a heterogeneous biological sample containing fetal DNA and high molecular non-fetal DNA such that the PEG or PEG-like polymer precipitates substantially the high molecular non-fetal DNA; and
purifying the fetal DNA from supernatant, thereby enriching the fetal DNA.
